(19) United States
(12) Patent Application Publication
(10) Pub. No.: US 2010/0068817 A1
(43) Pub. Date: Mar. 18, 2010

(54) COLORIMETRIC DETECTION OF METALLIC IONS IN AQUEOUS MEDIA USING FUNCTIONALIZED NANOPARTICLES

(75) Inventors: Chad A. Mirkin, Wilmette, IL (US); Jae-Seung Lee, Seongbuk-gu (KR); Min Su Han, Evanston, IL (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: NORTHWESTERN UNIVERSITY, EVANSTON, IL (US)

(21) Appl. No.: 12/513,246

(22) PCT Filed: Nov. 8, 2007
(86) PCT No.: PCT/US07/84026

§ 371 Date: May 1, 2009

Related U.S. Application Data
(60) Provisional application No. 60/857,599, filed on Nov. 8, 2006.

(51) Int. Cl. G01N 33/20

(52) U.S. Cl. 436/80; 436/81; 436/84

(57) ABSTRACT

Disclosed herein are methods of detecting metal ions in a sample using functionalized nanoparticles. More specifically, functionalized nanoparticles are used to selectively detect metal ions in a sample using changes in melting temperature of hybridized oligonucleotides on functionalized nanoparticles. The melting temperature can be detected using absorbance, color change, or both. In some cases, the concentration of the metal ion in the sample can be determined. Concentrations of metal ion of as little as 20 ppb (about 100 nM) can be detected using the disclosed methods.

### CROSS-REFERENCE TO RELATED APPLICATIONS

This application claims the benefit of U.S. Provisional Application Ser. No. 60/857,599, filed Nov. 8, 2006, which is incorporated herein in its entirety by reference.

### STATEMENT OF GOVERNMENT INTEREST

This invention was made with U.S. government support under National Science Foundation (NSF-NSEC) Grant No. EEC-011-8025 and Air Force Office of Scientific Research Grant No. F49620-01-1-04-01. The government has certain rights in this invention.

### BACKGROUND

The rapid determination of trace metals in biological and environmental systems is increasingly important in identifying potential hazards and preserving the public health. The toxicity of certain metals such as mercury, lead, and chromium is well-known.

The absorption of even trace amounts of lead can cause severe damage to human organs. The numerous and widespread sources of lead in the environment, including the food supply, compounds the problems of screening affected groups. It is generally recognized that lead poisoning occurs in children at blood levels as low as 10-15 μg/dl. Lead contamination of environmental sources such as water, dust and soil require identification at even lower levels. To measure these amounts, the analytical techniques must be sensitive, contaminant-specific, and reliable.

Mercury is a widespread pollutant with distinct toxicological profiles, and it exists in a variety of different forms (metallic, ionic, and as a component of organic and inorganic salts and complexes). Solvated mercuric ion, Hg²⁺, one of the most stable inorganic forms of mercury, is a caustic and carcinogenic material with high cellular toxicity (World Health Organization, Environmental Health Criteria 118: Inorganic Mercury, World Health Organization, Geneva, Switzerland (1991); Sekowski, et al., Toxicol. Appl. Pharmacol. 145: 268 (1997); Baum, Curr. Opin. Pediatr. 1999, 11, 265; J. -S. Chang, J. Hong, O. A. Ogunseitan, B. H. Olson, Biotechnol. Prog. 1993, 9, 526). The most common organic source of mercury, i.e., methyl mercury, can accumulate in the human body through the food chain and cause serious and permanent damage to the brain with both acute and chronic toxicity (Tchounwou, et al., Environ. Toxicol. 18:149 (2003); Clarkson, et al., N. Engl. J. Med. 349:1731 (2003); Morel, et al., Annu. Rev. Ecol. Syst. 29:543 (1998); Harris, et al., Science 301:1203 (2003); Boening, Chemosphere 40:1335 (2000))). Methyl mercury is generated by microbial biomethylation in aquatic sediments from water-soluble mercuric ion (Hg²⁺). Therefore, the ability to routinely detect Hg²⁺ is central to the environmental monitoring of rivers and larger bodies of water and for evaluating the safety of the aquatically-derived food supply (Brummer, et al., Bioorg. Med. Chem. 9:1067 (2001); Yoon, et al., J. Am. Chem. Soc. 127:16030 (2005)). Several methods for the detection of Hg²⁺, based upon organic fluorophores (Prodi, et al., J. Am. Chem. Soc., 122:6769 (2000); Nolan, et al., J. Am. Chem. Soc. 125:14270 (2003); Yang, et al., J. Am. Chem. Soc. 127:16760 (2005); Ros-Lis, et al., Angew. Chem. Int. Ed. 44:4405 (2005); Ono, et al., Angew. Chem. Int. Ed. 43:4300 (2004); Zhu, et al., Angew. Chem. Int. Ed. 45:3150 (2006); Guo, et al., J. Am. Chem. Soc. 126:2272 (2004); Caballero, et al., J. Am. Chem. Soc. 127:15666 (2005); Mello, et al., J. Am. Chem. Soc. 127:10124 (2005); Moon, et al., J. Org. Chem. 69:181 (2004); Wang, et al., J. Org. Chem. 71:4308 (2006); Sasaki, et al., Chem. Commun. 1581 (1998)) or chromophores, (Coronado, et al., J. Am. Chem. Soc., 127:12351 (2005); Nazeeruddin, et al., Adv. Funct. Mater., 16:189 (2006); Ros-Lis, et al., Inorg. Chem., 43:5183 (2004); Brummer, et al., Org. Lett., 1:415 (1999); Balaji, et al., Analyst, 130:1162 (2005); Huang, et al., J. Org. Chem., 70:5827 (2005); Palomares, et al., Chem. Commun., 362 (2004); Tatay, et al., Org. Lett., 8:3857 (2006)) semiconductor nanocrystals, (Chen, et al., Chem. Lett., 33:1608 (2004); Zhu, et al., Chem. Lett., 34:898 (2005)); cyclic voltammetry, (Nolan, et al., Anal. Chem., 71:3567 (1999); Kim, et al., Electroanalysis, 10:303 (1998)); polymeric materials, (Fan, et al., Macromolecules, 38:2844 (2005); Zhao, et al., J. Am. Chem. Soc,. 128:9988 (2006)) and microcantilevers, (Xu, et al., Anal. Chem., 74:3611 (2002)) have been developed. Colorimetric methods, in particular, are extremely attractive because they can be easily readout with the naked eye, potentially at the point-of-use. Although there are now several synthetic chromophores for Hg²⁺ based on the high thiophilicity of Hg²⁺ that provide simple colorimetric readout, all are limited with respect to sensitivity (i.e., current limit of detection is about 1 μM) and selectivity, kinetic instability, or incompatibility with aqueous environments.

Chromium and its compounds are primarily used in the manufacture of steel and other alloys, chrome plating, pigment production and leather tanning. In addition, chromate salts have been used for many years as excellent reagents in chemical laboratories. In the past, the hazardous characteristics of chromate compounds were not adequately recognized, such that chromium-containing waste often was inadequately disposed. At present, leaching of chromium compounds from waste sites to ground water has caused water contamination around the world. Drinking water contamination has been reported in many places in the

U.S. Chromium can exist in nature as a compound in one of two stable valences. Chromium in trivalent chromium (Cr³⁺) compounds is nontoxic and is actually an essential nutrient for the human body. Chromium in Cr⁶⁺ compounds is known to be carcinogenic. Therefore, chromium contamination is actually a problem of Cr⁶⁺ contamination. In water contamination investigations and contamination control, the concentration of Cr⁶⁺ in the water is of importance.

Recently, oligonucleotide-functionalized gold nanoparticles (Au NPs) have been used in a variety of forms for the detection of proteins (Nam, et al., Science, 301:1884 (2003); Georganopoulou, et al., Proc. Nat. Acad. Sci., 102:2273 (2005); and Niemeyer, Angew. Chem. Int. Ed., 40:4128 (2001)), oligonucleotides (Mirkin, et al., Nature, 382:607 (1996); Elghanian, et al., Science, 277:1078 (1997); Storhoff, et al., J. Am. Chem. Soc., 120:1959 (1998); Reynolds, et al., J. Am. Chem. Soc., 122:3795 (2000); Storhoff, et al., J. Am. Chem. Soc., 122:4640 (2000); Reynolds, et al., Pure. Appl. Chem., 72:229 (2000); Rosi, et al., Chem. Rev., 105:1547 (2005); Nam, et al., J. Am. Chem. Soc., 126:5932 (2004)), certain metal ions (Liu, et al., J. Am. Chem. Soc., 126:12298 (2004); Liu, et al., J. Am. Chem. Soc., 127:12677 (2005); Lin, et al., Angew. Chem. Int. Ed., 45:4948 (2006)), and other small molecules (Han, et al., J. Am. Chem. Soc., 128:4954 (2006); Liu, et al., Angew. Chem. Int. Ed., 45:90 (2006); Nam, et al., Anal. Chem., 77:6985 (2005); Han, et al., Angew. Chem. Int. Ed., 45:1807 (2006)). Functionalized Au NPs have characteristic high extinction coefficients (about 3 to 5 orders of magnitude higher than that of organic dye molecules—Yguerabide, et al., Anal. Biochem., 262:137 (1998)) and unique distance-dependent optical properties that can be chemically programmed through the use of specific oligonucleotide interconnects, which allows detection, in certain cases, of targets of interest through colorimetric means. Moreover, these structures, when hybridized to complementary particles, exhibit extremely sharp melting transitions, which have been used to enhance the selectivity of detection systems based upon them. Using such an approach, nucleic acid targets typically can be detected in the low nanomolar to high picomolar target concentration regimes in colorimetric format. Being able to use such particles to detect metal ions, such as copper, lead, chromium, and mercury, in the nanomolar concentration regime in colorimetric format would be a significant advance in the art.

### SUMMARY

Disclosed herein are methods of detecting metal ions in a sample. More specifically, disclosed herein are methods of detecting a metal ion of interest in a sample by comparing a melting temperature of a complex of a first functionalized nanoparticle and a second functionalized nanoparticle in the presence of the sample to a melting temperature of the same complex in the absence of the sample, where the complex has a nucleic acid motif selective for the metal ion of interest. Detection of the melting temperature can be via absorbance measurements and/or via detection of a color change. In various embodiments, the nanoparticle is a gold nanoparticle having a diameter of about 15 nm to about 250 nm. In some embodiments, the concentration of the metal ion in the sample can be as low as 100 nM (or about 20 ppb). In various embodiments, the metal ion is selected from the group consisting of silver, copper, and mercury. In certain embodiments, the nucleic acid motif is selected from the group consisting of a 8-hydroxyquinoline-8-hydroxyquinoline motif, a thymidine-thymidine mismatch, 6-(2′pyridyl)-purine nucleic acid motif, phenylenediamine motif, and a 2,6-bis(ethylthiomethyl)-3-pyridyl nucleic acid-2,6-bis(ethylthiomethyl)-3-pyridyl nucleic acid motif.

### BRIEF DESCRIPTION OF THE DRAWINGS

FIG. 1 shows a schematic of detection of mercuric ions using the disclosed methods, via a difference of melting temperature of aggregates of functionalized nanoparticles in the presence and absence of the mercuric ion.

FIG. 2A shows the normalized melting curve of aggregates of two complementary functionalized nanoparticles in the presence of different amounts of mercuric

FIG. 2B shows a graph of melting temperatures for the aggregates as a function of mercuric ion concentration.

FIG. 3A shows the normalized melting curve of aggregates of two complementary functionalized nanoparticles in the presence of different metal ions, each at a concentration of 1 μM, where 1. blank (no metal ion present); 2. Hg²⁺; 3. Li⁺; 4. Cd²⁺; 5. Ca²⁺; 6. Ba²⁺; 7. Mn²⁺; 8. Mg²⁺; 9. Zn²⁺; 10. Ni²⁺; 11. Fe²⁺; 12. Co²⁺; 13. Fe³⁺; 14. K⁺; 15. Cr³⁺; 16. Cu²⁺, and 17. Pb²⁺.

FIG. 3B shows the difference in melting temperature of the aggregates in the presence of the different metal ions.

FIG. 3C shows the color change of the aggregates in the presence of the different metal ions at 47° C.

### DETAILED DESCRIPTION

Disclosed herein are methods of detecting metal ions in a sample. More specifically, methods of detecting a metal ion of interest using functionalized nanoparticles are disclosed. Oligonucleotide functionalized nanoparticles are used due to a metal ion's ability to recognize and selectively bind to certain oligonucleotide motifs. For example, mercuric ion forms thymine-Hg²⁺-thymine complexes (Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954)). Copper ion forms 8-hydroxyquinoline (HQ)—copper-HQ complexes (Zhang, et al., J. Am. Chem. Soc., 127:74-75 (2005)). Silver ion forms a complex with 2,6-bis(ethylthiomethyl)-3-pyridine nucleic acid derivatives (Zimmermann, et al., J. Am. Chem. Soc., 124:13684-13685 (2002)).

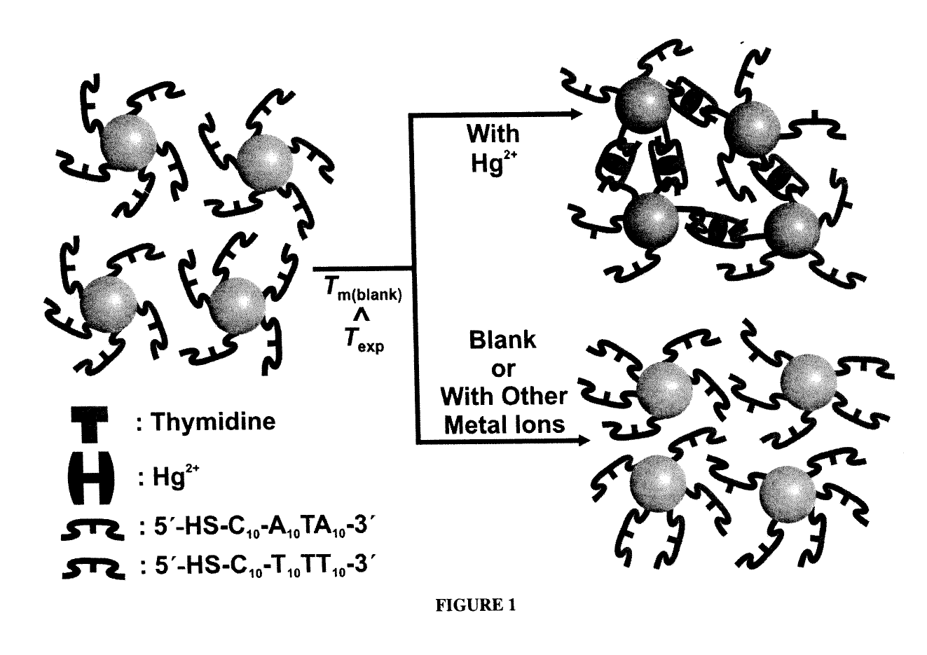

Thus, a method of detecting metal ions capable of binding to nucleic acid motifs is disclosed herein. Detection of the metal ion in the sample can comprises heating a complex comprising the sample and a first functionalized nanoparticle and a second nanoparticle, such that the first functionalized nanoparticle has a first oligonucleotide and the second functionalized nanoparticle has a second oligonucleotide, wherein the first oligonucleotide and the second oligonucleotide are sufficiently complementary to hybridize and, when hybridized, form a nucleic acid motif to which the metal ion can bind. The binding of the metal ion increases the melting point of the hybridized oligonucleotides. By comparing the melting temperature of the hybridized first oligonucleotide and second oligonucleotide on the functionalized nanoparticles in the presence and absence of the sample containing the metal ion, one can determine whether the metal ion is present and/or the concentration of the metal ion in the sample. This is outlined in FIG. 1, with Hg²⁺ as the metal ion and a thymine-thymine mismatch as the nucleic acid motif.

As seen in FIG. 1, the two functionalized nanoparticle form complexes in the presence of the metal ion, here mercuric ion, having a higher melting temperature than in the absence of the metal ion. By comparing the melting temperature of the hybridized complex in the presence and absence of the sample, one can determine if, and in certain cases, how much metal ion is in the sample. Thus, in some embodiments, the methods disclosed herein are qualitative, such that the presence of metal ion of interest is detected. In various cases, the methods are quantitative, such that the concentration of the metal ion of interest can be calculated based upon the observed difference in melting temperature.

As used herein, the term “sample” refers to biological or environmental samples. Biological samples include, but are not limited to, a fluid such as urine, blood, plasma, serum, saliva, semen, stool, sputum, cerebral spinal fluid, tears, mucus, and the like. Biological samples can be from human or animal. Environmental samples include, but are not limited to, soil and water, such as groundwater.

As used herein, the term “metal ion” refers to any metal ion in any oxidation state which may be found in an environmental or biological sample and can bind to a nucleic acid motif. Nonlimiting examples include mercury, copper, silver, nickel, and palladium.

As used herein, the term “nucleic acid motif” refers to an alignment of functional groups of nucleobases in a hybridized oligonucleotide structure which is sufficient to allow for metal ion binding. Preferably, the nucleic acid motif is specific for a particular metal ion, such that the metal ion of interest is the predominant metal to bind to the nucleic acid motif. By predominant is meant that the majority of the metal ion that binds to the nucleic acid motif is the metal ion of interest. In some embodiments, the metal ion of interest binds 5 times more, 7 times more, 10 times more, 20 times more, 25 times more, 30 times more, 40 times more, 50 times more, 100 times more, 200 times more, 300 times more, 400 times more, 500 times more, or 1000 times more than any one other metal ion.

The nucleic acid motif can comprise natural nucleobases, synthetic nucleobases, or a mixture thereof. Specific, nonlimiting, examples of metal ions bound to nucleic acid motifs are depicted below in Scheme 2.

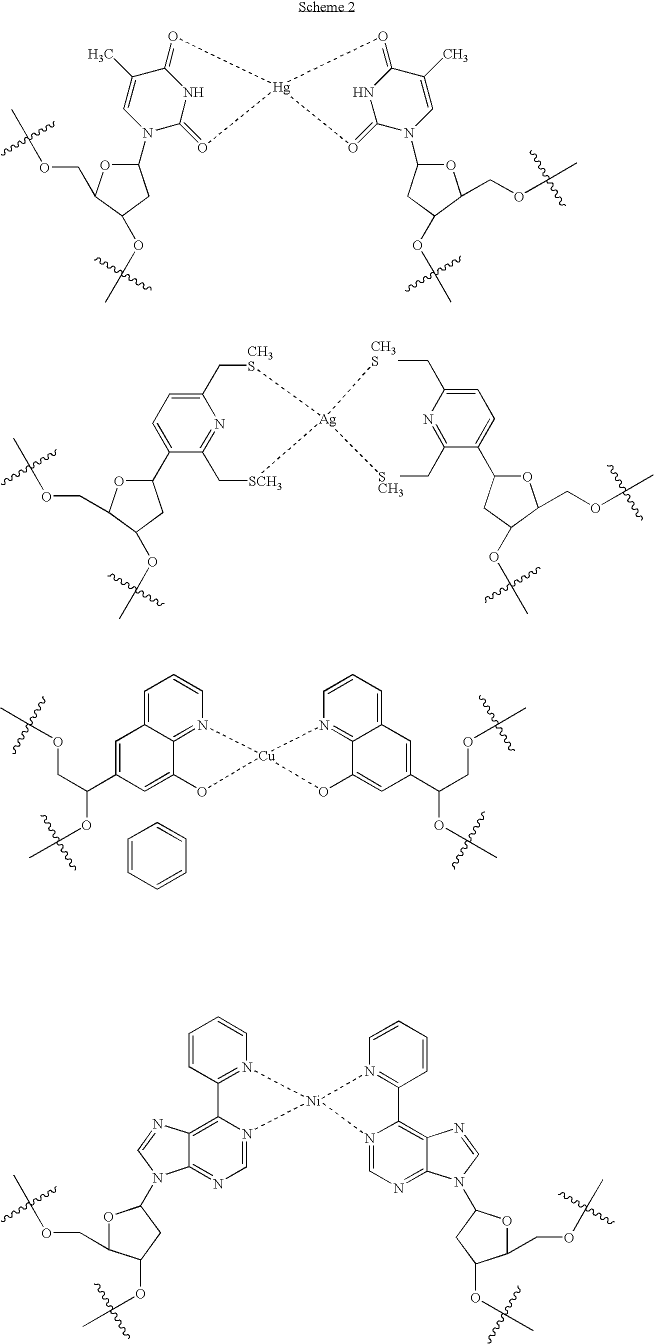

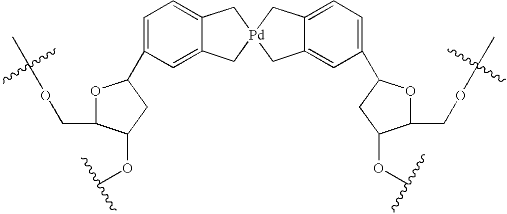

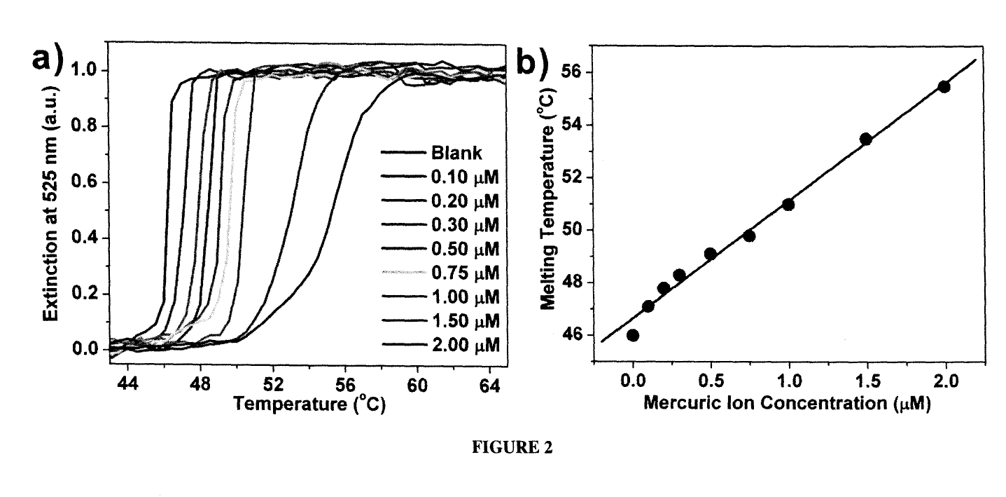

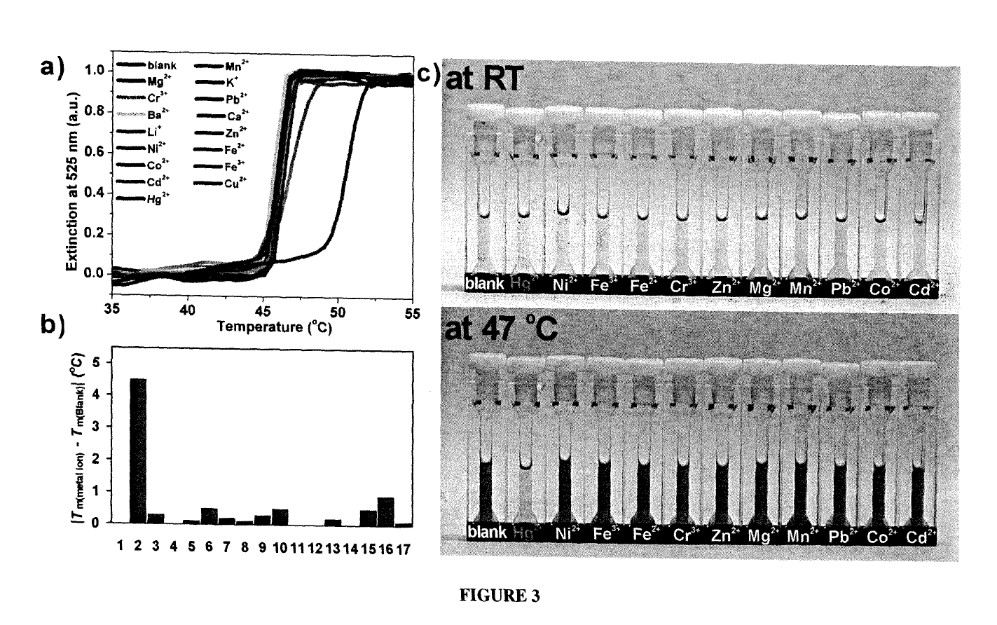

The binding of the metal ion to the nucleic acid motif of the hybridized oligonucleotides on two functionalized nanoparticles increases the melting temperature of the hybridized oligonucleotides. Thus, the presence of a metal ion will result in a higher melting temperature, which can be spectroscopically, and in certain cases, visually, detected. Absorbance of the functionalized nanoparticles can be monitored at 525 nm, where gold nanoparticles have maximum intensity. The absorbance is decreased when the nanoparticles are hybridized to other nanoparticles. When the oligonucleotides melt, an increase in absorbance results. This is seen in FIG. 2A, where as the temperature increases, the absorbance increases at the melting temperature of the hybridized oligonucleotides. This melting can also be seen as a color change from colorless or pale purple to a dark red when the hybridized oligonucleotides on the functional nanoparticles melt. This color change can be observed in, e.g., FIG. 3C, described in detail below.

In some cases, the methods disclosed herein can be used to detect a metal ion in a sample, where the sample also contains a second metal ion. The method can selectively detect the metal ion of interest in the presence of the second metal ion. This principle is demonstrated in FIG. 3, where mercuric ion is detected via a change in melting temperature, while other metal ions are show no change in melting temperature.

In various cases, the methods disclosed herein can be used to determine the concentration of the metal ion in solution. The change in melting temperature can be correlated to the concentration of the metal ion. Thus, a comparison of the melting temperature of a sample having a metal ion of unknown concentration to a standard curve of melting temperatures of known concentration of metal ion can provide the concentration of the metal ion in the sample.

### Functionalized Nanoparticles

Functionalized nanoparticles are used in the disclosed methods. The term “functionalized nanoparticle,” as used herein, refers to a nanoparticle having at least a portion of its surface modified with an oligonucleotide. In one embodiment, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the invention include, also without limitation, ZnS, ZnO, Ti, TiO₂, Sn, SnO₂, Si, SiO₂, Fe, Ag, Cu, Ni, Al, steel, cobalt-chrome alloys, Cd, titanium alloys, AgI,

AgBr, HgI₂, PbS, PbSe, ZnTe, CdTe, In₂S₃, In₂Se₃, Cd₃P₂, Cd₃As₂, InAs, and GaAs. Methods of making ZnS, ZnO, TiO₂, AgI, AgBr, HgI₂, PbS, PbSe, ZnTe, CdTe, In₂S₃, In₂Se₃, Cd3P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshaysky, et al., J. Am. Chem. Soc., 112, 9438 (1990); and Ushida et al., J. Phys. Chem., 95, 5382 (1992).

In practice, methods are provided using any suitable nanoparticle having oligonucleotides attached thereto having a suitable nucleic acid motif and that are in general suitable for use in the disclosed detection assays, which do not interfere with oligonucleotide complex formation, i.e., hybridization to form a double-strand complex. The size, shape and chemical composition of the particles contribute to the properties of the resulting oligonucleotide-functionalized nanoparticle. These properties include for example, optical properties, optoelectronic properties, electrochemical properties, electronic properties, stability in various solutions, magnetic properties, and pore and channel size variation. The use of mixtures of particles having different sizes, shapes and/or chemical compositions, as well as the use of nanoparticles having uniform sizes, shapes and chemical composition, is contemplated. Examples of suitable particles include, without limitation, nanoparticles, aggregate particles, isotropic (such as spherical particles) and anisotropic particles (such as non-spherical rods, tetrahedral, prisms) and core-shell particles, such as those described in U.S. Pat. No. 7,238,472 and International Publication No. WO 2003/08539, the disclosures of which are incorporated by reference in their entirety.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers). Suitable nanoparticles are also commercially available from, for example, Ted Pella, Inc. (gold), Amersham Corporation (gold) and Nanoprobes, Inc. (gold). Tin oxide nanoparticles having a dispersed aggregate particle size of about 140 nm are available commercially from Vacuum Metallurgical Co., Ltd. of Chiba, Japan. Other commercially available nanoparticles of various compositions and size ranges are available, for example, from Vector Laboratories, Inc. of Burlingame, Calif.

Also, as described in U.S. patent publication No 2003/0147966, nanoparticles comprising materials described herein are available commercially, or they can be produced from progressive nucleation in solution (e.g., by colloid reaction) or by various physical and chemical vapor deposition processes, such as sputter deposition. See, e.g., HaVashi, Vac. Sci. Technol. A5(4):1375-84 (1987); Hayashi, Physics Today, 44-60 (1987); MRS Bulletin, January 1990, 16-47. As further described in U.S. patent publication No 2003/0147966, nanoparticles contemplated are produced using HAuCl₄ and a citrate-reducing agent, using methods known in the art. See, e.g., Marinakos et al., Adv. Mater. 11:34-37(1999); Marinakos et al., Chem. Mater. 10: 1214-19(1998); Enustun & Turkevich, J. Am. Chem. Soc. 85: 3317(1963).

Methods of making oligonucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically. Non-naturally occurring nucleobases can be incorporated into the oligonucleotide, as well. See, e.g., Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

At least one oligonucleotide is bound to the nanoparticle through a 5′ linkage and/or the oligonucleotide is bound to the nanoparticle through a 3′ linkage. In various aspects, at least one oligonucleotide is bound through a spacer to the nanoparticle. In these aspects, the spacer is an organic moiety, a polymer, a water-soluble polymer, a nucleic acid, a polypeptide, and/or an oligosaccharide. Methods of functionalizing the oligonucleotides to attach to a surface of a nanoparticle are well known in the art. See Whitesides, Proceedings of the Robert A. Welch Foundation 39th Conference On Chemical Research Nanophase Chemistry, Houston, Tex., pages 109-121 (1995). See also, Mucic et al. Chem. Comm. 555-557 (1996) (describes a method of attaching 3′ thiol DNA to flat gold surfaces; this method can be used to attach oligonucleotides to nanoparticles). The alkanethiol method can also be used to attach oligonucleotides to other metal, semiconductor and magnetic colloids and to the other nanoparticles listed above. Other functional groups for attaching oligonucleotides to solid surfaces include phosphorothioate groups (see, e.g., U.S. Pat. No. 5,472,881 for the binding of oligonucleotide-phosphorothioates to gold surfaces), substituted alkylsiloxanes (see, e.g. Burwell, Chemical Technology, 4:370-377 (1974) and Matteucci and Caruthers, J. Am. Chem. Soc., 103:3185-3191 (1981) for binding of oligonucleotides to silica and glass surfaces, and Grabaretal., Anal. Chem., 67:735-743 for binding of aminoalkylsiloxanes and for similar binding of mercaptoaklylsiloxanes). Oligonucleotides terminated with a 5′ thionucleoside or a 3′ thionucleoside may also be used for attaching oligonucleotides to solid surfaces. The following references describe other methods which may be employed to attached oligonucleotides to nanoparticles: Nuzzo et al., J. Am. Chem. Soc., 109:2358 (1987) (disulfides on gold); Allara and Nuzzo, Langmuir, 1:45 (1985) (carboxylic acids on aluminum); Allara and Tompkins, J. Colloid Interface Sci., 49:410-421 (1974) (carboxylic acids on copper); Iler, The Chemistry Of Silica, Chapter 6, (Wiley 1979) (carboxylic acids on silica); Timmons and Zisman, J. Phys. Chem., 69:984-990 (1965) (carboxylic acids on platinum); Soriaga and Hubbard, J. Am. Chem. Soc., 104:3937 (1982) (aromatic ring compounds on platinum); Hubbard, Acc. Chem. Res., 13:177 (1980) (sulfolanes, sulfoxides and other functionalized solvents on platinum); Hickman et al., J. Am. Chem. Soc., 111:7271 (1989) (isonitriles on platinum); Maoz and Sagiv, Langmuir, 3:1045 (1987) (silanes on silica); Maoz and Sagiv, Langmuir, 3:1034 (1987) (silanes on silica); Wasserman et al., Langmuir, 5:1074 (1989) (silanes on silica); Eltekova and Eltekov, Langmuir, 3:951 (1987) (aromatic carboxylic acids, aldehydes, alcohols and methoxy groups on titanium dioxide and silica); Lec et al., J. Phys. Chem., 92:2597 (1988) (rigid phosphates on metals).

The length of the oligonucleotide on the nanoparticle surface is typically about 15 to about 100 nucleobases. Less than 15 nucleobases can result in a oligonucleotide complex having a too low a melting temperature to be suitable in the disclosed methods. More than 100 nucleobases can result in a oligonucleotide complex having a too high melting temperature to be suitable in the disclosed methods. Thus, oligonucleotides of about 15 to about 100 nucleobases are preferred. The oligonucleotide length can be about 20 to about 70, about 22 to about 60, or about 25 to about 50 nucleobases.

Two differently functionalized nanoparticles are employed in the methods disclosed herein, each nanoparticle having a different, but at least partially complementary, oligonucleotide on its surface. Thus, the first functionalized nanoparticle comprises a first oligonucleotide on at least a portion of the surface of the first nanoparticle and the second functionalized nanoparticle comprises a second oligonucleotide on at least a portion of the surface of the second nanoparticle. The first and second oligonucleotides are complementary and are typically at least about 50% complementary, but can be at least about 60%, at least about 70%, at least about 80%, or at least about 90% complementary.

### Metal Ion Detection Using Nanoparticles

Three components of the disclosed methods contribute to the high sensitivity, selectivity, and quantitative aspects of the invention: (1) the oligonucleotides, (2) the Au NPs, and (3) the oligonucleotide-nanoparticle conjugate. The chelating ability of the nucleic acid motif that form in the hybridized oligonucleotides of the functionalized nanoparticles is selective for the metal ion. For example, it is known that two thymidine residues when geometrically pre-organized in a DNA duplex can behave as a chelate and form a tightly bound complex with Hg²⁺ (Miyake, et al., J. Am. Chem. Soc., 128:2172 (2006)). The high extinction coefficients of Au NPs (about 109 cm⁻¹ M⁻¹ for 15 nm Au NPs) can act as an amplifier for the permutation of the Tₘ upon binding Hg²⁺, allowing ppb detection limits. Conventional chromogenic chemosensors have relatively low extinction coefficients (typically about 105 cm⁻¹ M⁻¹), which limit their sensitivity to the sub-micromolar concentration range at best. The sharp, highly cooperative melting properties of oligonucleotide-Au NP conjugates enable one to distinguish subtle Tₘ differences, providing exquisite measure of the Hg²⁺ concentration over the 100 nM to micromolar concentration range.

### Examples

### Detection of Mercuric Ion Using Functionalized Nanoparticles

Two types of gold nanoparticles (Au NPs, designated as probe A and probe B) were prepared, each functionalized with different thiolated-DNA sequences (probe A: 5′ HS-C₁₀-A₁₀-T-A₁₀ 3′—SEQ ID NO. 1; probe B: 5′ HS-C₁₀- T₁₀-T-T₁₀ 3′—SEQ ID NO. complementary except for a single thymidine-thymidine mismatch (see Scheme 1). Gold nanoparticles (Au NP—15 nm) were purchased from Ted Pella, and used as received.

Oligonucleotides (5′-modified) were synthesized on a 1 μmol scale using an automated synthesizer (Milligene Expedite) following the standard protocol for phosphoramidite chemistry and purified by HPLC (HP 1100 system). All of the reagents required for the oligonucleotide synthesis were purchased from Glen Research (Sterling, Va.). For the preparation of DNA-Au NPs, the terminal disulfide groups of the DNA strands were reduced by soaking it in a 0.1 M dithiothreitol phosphate buffer solution (0.17 M phosphate, pH 8.0) for 30 min. The cleaved DNA strands were purified by NAP-5 column (GE Healthcare) and added to the gold colloid (at a final oligonucleotide concentration of about 3 μM). The solution was buffered to 0.15 M sodium chloride (NaCl), 10 mM phosphate, and 0.01% sodium dodecyl sulfate (SDS) by simultaneously adding appropriate amount of 1% SDS solution, 2 M NaCl solution and 0.1 M phosphate buffer solution (pH 7.4). After the incubation overnight at room temperature with gentle shaking, the Au NP solution was centrifuged and redispersed in 0.1 M sodium nitrate (NaNO₃), 0.005% Tween 20, 10 mM MOPS buffer (detection buffer, pH 7.5) after the supernatant was removed. The particles were washed three times more, and finally redispersed in the detection buffer. Probe A and probe B (1.5 pmol each) were mixed, incubated overnight at 4° C. to form aggregates, and stored until use.

Upon hybridization, the probe A-modified Au NP and probe B-modified Au NP form stable aggregates and exhibit characteristic sharp melting transitions (full width at half maximum less than about 1° C.) associated with aggregates formed from perfectly complementary particles, but with a lower Tₘ. (See, e.g., Rentzeperis, et al., Biochemistry, 34, 2937-2945 (1995); Coll, et al. Proc. Natl. Acad. Sci. USA, 84, 8385-8389 (1987).) It was hypothesized that Hg²⁺ would selectively bind to thymidine-thymidine mismatch sites in the aggregates formed from mismatched strands and raise the Tₘ of the resulting structures since it is known that Hg²⁺ coordinates selectively to thymidine (Katz, J. Am. Chem. Soc. 74:2238 (1951); Yamane, et al., J. Am. Chem. Soc, 83:2599 (1961); Kosturko, et al., Biochemistry, 13:3949 (1974); Thomas, J. Am. Chem. Soc., 76:6032 (1964); Miyake, et al., J. Am. Chem. Soc., 128:2172 (2006)). The analogous interaction with particle-free DNA leads to significant increases in Tₘ of about 10° C.

An aliquot of an aqueous solution of Hg²⁺ at a designated concentration was added to a solution of the aggregates formed from probes A and B (where probes A and B were initially at 1.5 nM each) at room temperature. The solution then was heated at a rate of 1° C./min while its absorbance was monitored at 525 nm where the Au NP probes have the maximum intensity. The Tₘ was obtained at the maximum of the first derivative of the melting transition. Without Hg²⁺, the aggregates melt with a dramatic purple-to-red color change at about 46° C. In the presence of Hg²⁺, however, the aggregate melted at temperatures higher than 46° C. due to the strong coordination of Hg²⁺ to two thymidines from different strands (e.g., probes A and B), thereby stabilizing the duplex DNA containing the T-T single base mismatch.

### Determination of Concentration of Mercuric Ion in a Sample

To evaluate the sensitivity of the assay, different concentrations of Hg²⁺ from one stock solution were tested. When an Hg²⁺ sample was mixed with the Au NP probe aggregate solution, there was no noticeable change under the reaction conditions described above. Once heated, however, the aggregates melted with a significant color change at a specific temperature (FIG. 1A) linearly related to the concentration of Hg²⁺ over the entire range of detectable Hg²⁺ studied (FIG. 1B). The limit of detection for this system was about 100 nM, which is about 20 ppb, Hg²⁺ (FIG. 1A). Each increase in concentration of 1 μM resulted in about a 5° C. increase in Tₘ, providing a way of determining Hg²⁺ concentration in a sample.

The colorimetric detection of Hg²⁺ was performed by mixing the Hg²⁺ stock solution, the aggregates of Au NP probes prepared as described above, and the detection buffer to the final volume of 1 mL at room temperature. The final concentration of the Au NP probes was 3 nM in total. Hg²⁺ stock solution was prepared by dissolving Hg(ClO₄)2.xH₂O (Sigma-Aldrich) in the detection buffer. Melting transition of the mixture was obtained shortly thereafter by monitoring the absorbance at 525 nm as a function of temperature at a rate of 1° C. per min (Cary 500, Varian).

### Selectivity for a Metal Ion

The selectivity of the system described above for Hg²⁺ was evaluated by testing the response of the assay to other environmentally relevant metal ions including Mg²⁺, Pb²⁺, Cd²⁺, Co²⁺, Zn²⁺, Fe²⁺, Ni²⁺, Fe³⁺, Mn²⁺, Ca²⁺, Ba²⁺, Li⁺, K⁺, Cu²⁺, and Cr³⁺ (FIGS. 2A and 2B) at a concentration of 1 μM, from their perchlorate salts. The melting of aggregates without any metal ion (blank) was also monitored as a control experiment. Only the Hg²⁺ sample showed a significantly higher Tₘ (ΔTₘ of about 5° C.) compared to that of the blank. Pb²⁺ elevates the Tₘ a negligible amount (ΔTₘ of about 0.8° C.). Importantly, sensors that can detect analytes with the naked eye without resorting to any instrument are of particular interest because their simplicity and convenience. A typical demonstration of the colorimetric assay presented in this work (FIG. 2C) showed that at 47° C., the color of the aggregate solution in the presence of Hg²⁺ is not changed, but the color of the other aggregate solutions are dramatically changed from pale purple to dark red.

### Stability of Functionalized Nanoparticles in Presence of Metal Ion

It is important to keep a consistent number of DNA strands per particle throughout the entire assay procedure because thiophilic Hg²⁺ can possibly detach the thiolated DNA strands from the Au NP surface by forming Hg²⁺-thiol complex structures. This displacement during the assay would result in the loss of detection accuracy. To verify that nanoparticle probes are functional and stable in the presence of Hg²⁺, the number of DNA strands per particle at various concentrations of Hg²⁺ for a elongated time period was measured using fluorophore-labeled DNA (5′ HS-C₁₀-A₁₀-T-A₁₀-(6-FAM) 3′—SEQ ID NO. 3). No quenching effect of Hg²⁺ on the fluorophore was observed, and the initial number of the fluorophore-labeled DNA strands per particle has been determined to be 70 by fluorescence studies using dithiothreitol (DTT) as a oligonucleotide stripping reagent. The fluorophore-labeled DNA-functionalized Au NPs which remained in the presence of 0.5, 1, and 2 μM of Hg²⁺ for 8 hours at room temperature did not often lose any number of the strands (Table 1). The stability of the particles was confirmed by testing them under the same conditions except for the elevated temperature (50° C.), and the loss of DNA was still less than 10% of the total DNA strands regardless of the concentration of Hg²⁺, which was caused mainly by the higher temperature, not Hg²⁺ (Table 1). Therefore, no effect of the concentrations of Hg²⁺ tested was observed on the number of DNA strands per particle and the functionality of DNA-Au NPs even at higher temperature after a significantly extended time period.

TABLE 1

| | | Mercuric Ion Concentration | | |
|---|---|---|---|---|
| Temperature | Portion | 0.5 μM | 1 μM | 2 μM |
| Room Temperature | In the Supernatant | 2.1 | 1.6 | 1.8 |
| | On the Particles | 68.0 | 68.9 | 68.1 |
| 50° C. | In the Supernatant | 5.9 | 6.0 | 6.1 |
| | On the Particles | 64.7 | 64.1 | 65.0 |

For the fluorescence study, 15 nm Au NPs were functionalized with fluorophore-labeled DNA as described above. Au NPs (3 nM) were mixed with 0.5, 1 and 2 μM of Hg²⁺ for 8 hours at either room temperature or 50° C. The Au NP solutions were centrifuged and the supernatant was decanted for the analysis of the detached DNA strands. The Au NPs were washed 4 more times with the detection buffer by centrifugation and finally redispersed in 0.5 M DTT solution in the detection buffer for 1 hour to release the fluorophore-labeled DNA from the Au NPs. The released DNA was collected from the supernatant after centrifugation at 13,000 rpm for 10 min. The number of DNA strands per particle was calculated from the amount of DNA and the number of Au NPs.

What is claimed:

1. A method of detecting a first metal ion in a sample comprising:
a) heating a first complex comprising (1) a first functionalized nanoparticle and a second functionalized nanoparticle and (2) the sample containing or suspected of containing the first metal ion to determine a melting temperature of the first complex,
b) comparing the melting temperature of the first complex to a melting temperature of a second complex comprising the first functionalized nanoparticle and the second functionalized nanoparticle in the absence of the first metal ion, wherein when the first complex has a higher melting temperature than the second complex, the sample comprises the first metal ion; and
wherein the first functionalized nanoparticle comprises a first oligonucleotide on at least a portion of the nanoparticle surface and the second functionalized nanoparticle comprises a second oligonucleotide on at least a portion of the nanoparticle surface, and the first oligonucleotide and second oligonucleotide comprise a nucleic acid motif selective for the first metal ion and the first oligonucleotide is sufficiently complementary to the second oligonucleotide to hybridize.

2. The method of claim 1, further comprising heating the second complex to determine the melting temperature of the second complex.

3. The method of claim 1, wherein the first metal ion is selected from the group consisting of mercuric ion, copper ion, silver ion, nickel ion, and palladium ion.

4. The method of claim 1, wherein the first metal ion is mercuric ion and the nucleic acid motif comprises a thymine-thymine mismatch.

5. The method of claim 1, wherein the first metal ion is copper ion and the nucleic acid motif comprises a 8-hydroxyquinoline-8-hydroxyquinoline motif.

6. The method of claim 1, wherein the first metal ion is silver ion and the nucleic acid motif comprises a 2,6-bis(ethylthiomethyl)-3-pyridyl nucleic acid-2,6-bis(ethylthiomethyl)-3-pyridyl nucleic acid motif.

7. The method of claim 1, wherein the first metal ion is nickel ion and the nucleic acid motif comprises a 6-(2′pyridyl)-purine.

8. The method of claim 1, wherein the first metal ion is palladium ion and the nucleic acid motif comprises phenylenediamine.

9. The method of claim 1, further comprising calculating a concentration of the first metal ion in the sample by comparing the melting temperature of the first complex to a standard curve comprising melting temperatures of complexes in the presence of known concentrations of the first metal ion.

10. The method of claim 9, wherein the first metal ion has a concentration of at least about 100 nM.

11. The method of claim 1, wherein the first functionalized nanoparticle, second functionalized nanoparticle, or both comprises a gold nanoparticle having a diameter of about 13 nm to about 250 nm.

12. The method of claim 1, wherein the sample further comprises a second metal ion and wherein the method selectively detects the presence of the first metal ion.

13. The method of claim 1, wherein the melting temperature of the first complex, the second complex, or both is determined by a change in color of the first complex, second complex, or both.

14. The method of claim 1, wherein the melting temperature of the first complex, the second complex, or both is determined by monitoring the absorbance of the first complex, the second complex, or both.


SEQUENCE LISTING (3 sequences)

SEQ ID NO: 1 - DNA, 31 bp, Artificial sequence
  Synthetic polynucleotide
ccccccccccaaaaaaaaaataaaaaaaaaa

SEQ ID NO: 2 - DNA, 31 bp, Artificial sequence
  Synthetic polynucleotide
ccccccccccttttttttttttttttttttt

SEQ ID NO: 3 - DNA, 31 bp, Artificial sequence; identical to SEQ ID NO: 1
  Synthetic polynucleotide